(19) United States
(12) Patent Application Publication
(10) Pub. No.: US 2004/0018983 A1
(43) Pub. Date: Jan. 29, 2004

(54) Antifungal compositions

(75) Inventors: William G. Rice, Madison, CT (US); Douglas D. Buechter, Wallingford, CT (US); Randall B. Murphy, Irvington, NY (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Achillion Pharmaceuticals, Inc.

(21) Appl. No.: 10/327,239

(22) Filed: Dec. 20, 2002

Related U.S. Application Data
(60) Provisional application No. 60/343,417, filed on Dec. 21, 2001.

(51) Int. Cl. A61K 38/05; A61K 31/198; A61K 31/195; C12Q 1/18

(52) U.S. Cl. 514/18; 514/562; 435/32

(57) ABSTRACT

Methods for elucidating an antifungal or anti-yeast compound which selectively bind to a fungal or yeast zinc finger-containing protein within a fungus are disclosed. Assays of screening for compounds that are effective for binding to a fungal or yeast zinc finger-containing protein are also provided. It is also provided a pharmaceutical composition containing an effective amount of a compound or compounds identified as effective for binding to or associating with a fungal or yeast zinc finger-containing protein using the disclosed methods. Fungal and/or yeast infections can be treated or prevented by administering to a patient in need of treatment the pharmaceutical composition containing an effective amount of an compound or compounds identified as effective for binding to or associating with a fungal or yeast zinc finger-containing protein using the disclosed methods.

### CROSS REFERENCE TO RELATED APPLICATIONS

Priority is claimed to U.S. Provisional application Serial No. 60/343,417, filed Dec. 21, 2001, the teachings of which are incorporated herein.

### FIELD OF THE INVENTION

This application generally relates to the field of anti-fungal or anti-yeast compounds. Specifically, the application is drawn to compounds that bind to zinc finger-containing motifs within proteins thereby altering the biological function and structure of the protein. More specifically, the application relates to methods for determining the biological activity and chemical specificity of compounds that bind to or associate with zinc finger-containing motifs within proteins thereby altering the biological function and structure of the protein.

### BACKGROUND OF THE INVENTION

Systemic fungal infections in man are relatively rare in temperate countries. Many of the fungi that can become pathogenic normally live commensally in the body although they are common in the environment. However, the past few decades have witnessed an increasing incidence of numerous life-threatening systemic fungal infections worldwide. These diseases now represent a major threat to many susceptible patients, particularly those already hospitalized. Most of the increase can be attributed to improved survival of immunocompromised patients and the chronic use of antimicrobial agents. Moreover, the flora typical of many common fungal infections is also changing and this is presenting an epidemiological challenge of increasing importance. Patients at greatest risk include those with impaired immune functioning, either directly as a result of immunosuppression from cytotoxic drugs or HIV infection, or secondary to other debilitating diseases such as cancer, acute leukemia, invasive surgical techniques or prolonged exposure to anti-microbial agents.

The most common systemic fungal infections in man are candidosis, aspergillosis, histoplasmosis, coccidioidomycosis, paracoccidioidomycosis, blastomycosis and cryptococcosis. Fungi are eukaryotic in nature, as are mammalian cells, and thus typical interventive targets are often shared among the fungi and host cells. Upon drug treatment, this often leads to host cell toxicity and a general scarcity of effective, non-toxic antifungal agents. Currently utilized antifungals are primarily classified as polyenes, azoles or antimetabolites. Polyenes such as amphotericin B demonstrate affinity for ergosterol in fungal membranes, rather than the more abundant cholesterol in mammalian cell membranes. These generally broad-spectrum antifungal agents form leaky channels in fungal membranes. Unfortunately, drug-resistant isolates having modified membrane steroid are emerging and represent a significant clinical problem.

Antifungals such as ketoconazole, itraconazole and fluconazole (the azoles) are being increasingly employed for the treatment and prophylaxis of systemic fungal infections in immunocompromised patients. These azoles inhibit cytochrome-P450 activity and interfere with ergosterol synthesis and hence membrane integrity. Such compounds are fungistatic, not fungicidal, and are thus less clinically effective. Also, such compounds can affect mammalian Cyt-P450 activities and modify steroid synthesis in humans. However, concern about fungal resistance to some of these agents, especially the more narrow spectrum ones, e.g. fluconazole, is growing.

Antimetabolites, such as Flucytosine that converts to the 5-fluorouracil (5-FU) active metabolite that inhibits thymidylate synthase, can cause bone marrow depression, CNS toxicity and gastrointestinal distress. Moreover, drug resistance typically develops rapidly to such antimetabolites.

Worse still, it is recognized in the medical world that about 40% of the people suffering from severe systemic fungal infections are hardly able, or not at all able to receive medication via oral administration. This inability is due to the fact that such patients are in a coma or suffer from severe gastroparesis. Hence the use of insoluble or sparingly soluble antifungals such as itraconazole or saperconazole, that are difficult to administer intravenously, cannot be used for these patients.

Consequently, there is a need for new antifungals, preferably active against broad-spectrum antifungal targets that possess the following properties: 1) no cross-resistance with current antifungals that affect other targets, 2) structural distinctiveness compared to mammalian counterparts such that selectivity can be achieved toward the fungal target, and 3) structural and functional conservation so that mutational escape toward drug-resistance is minimized.

It is therefore an object of the present invention to provide methods to elucidate compounds to modulate the biological activity of one or more fungal or yeast zinc finger proteins.

It is a further object of the present invention to provide methods for screening for compounds that are effective as antifungal or anti-yeast agents.

### SUMMARY OF THE INVENTION

Screens have been designed that enable the determination of the effectiveness of agents to interact with zinc finger-containing proteins and alter the function of the fungal protein. These screens involve the use of a zinc finger-blocking compound, which is present in the assay screen at a concentration greater than the test compound is present. Most typically, the zinc finger-blocking compound is present in the assay medium at, for example, a 10-fold to 100-fold greater concentration than the test compound. In the presence of the zinc finger-blocking compound, reaction of a covalent nature with the zinc finger motif is prevented. The assay may involve the whole fungal organism, as the zinc finger blocking protein alone will not inhibit or facilitate fungal growth. This can be empirically determined in appropriate controls that are run at the same time as the assay procedure. The assay also allows the use of multiple zinc finger blocking compounds or combinations thereof. This may be desirable in certain types of fungal organisms where certain types of the zinc finger blocking compounds, or the test compounds, may be more or less accessible due to differences in the structure of the fungal membranes of said organisms.

Antifungal compositions for pharmaceutical use or agricultural use can be formed by combining one or more antifungal compounds with a pharmaceutically active carrier. Disorders to be treated or prevented by these compositions can be any disorders caused by one or multiple fungi, such as aspergillosis, blastomycosis, candidosis, chromo mycosis, coccidioidomycosis, cryptococcosis, histoplasmosis, paracoccidioidomycosis, phaeohyphomycosis, phycomycosis, pneumocystis carinii infection, pseudallescheria boydii infection, scedosporium apiospermum infection, sporotrichosis, dermatophytoses, Torulopsis infection, mucorales infection, sporothrix infection.

Effective dosages of the antifungal compositions disclosed herein can be determined on the basis of the in vitro or in vivo assay of the compounds tested against the appropriate fungus.

### DETAILED DESCRIPTION OF PREFERRED EMBODIMENTS

I. Definitions

The term “compound” as used herein means an antifungal compound, or a derivative thereof, which binds selectively to the zinc finger motif of a zinc finger-containing protein of a fungi or yeast. As used herein, the term “agent” and “active ingredient” are used interchangeably with the term “compound.”

The term “alkyl” means a straight or branched chain hydrocarbon. Representative examples of alkyl groups include methyl, ethyl, propyl, isopropyl, butyl, isobutyl, tert-butyl, sec-butyl, pentyl, and hexyl.

The term “alkoxy” means an alkyl group bonded to an oxygen atom. Representative examples of alkoxy groups include methoxy, ethoxy, tert-butoxy, propoxy, and isobutoxy. Preferred alkoxy groups are C1-C8 alkoxy.

The term “halogen” means fluorine, chlorine, bromine or iodine. The term “alkenyl” means a branched or straight chain hydrocarbon having one or more carbon-carbon double bonds.

The term “alkynyl” means a branched or straight chain hydrocarbon having one or more carbon-carbon triple bonds.

The term “cycloalkyl” means a cyclic hydrocarbon. Examples of cycloalkyl groups include cyclopropyl, cyclobutyl, cyclopentyl, cyclohexyl and cycloheptyl. Preferred cycloalkyl groups are C3-C8-cyloalkyl. It is also possible for the cycloalkyl group to have one or more double bonds, but is not aromatic. Examples of cycloalkyl groups having a double bond include cyclopentenyl, cyclohexenyl, cyclohexadienyl, cyclobutadienyl, and the like.

The term “perfluoroalkyl” means an alkyl group in which all of the hydrogen atoms have been replaced with fluorine atoms. The term “acyl” means a group derived from an organic acid (—COOH) by removal of the hydroxy group (—OH).

The term “aryl” means a cyclic, aromatic hydrocarbon. Examples of aryl groups include phenyl and naphthyl.

The term “heteroatom” includes oxygen, nitrogen, sulfur, and phosphorous.

The term “heteroaryl” means an aromatic ring containing one or more heteroatoms. If the heteroaryl group contains more than one heteroatoms, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six-membered rings and contain from one to three heteroatoms.

The term “heterocycloalkyl” mean a cycloalkyl group in which one or more of the carbon atoms have been replaced with a heteroatom. If the heterocycloalkyl group contains more than one heteroatom, the heteroatoms may be the same or different. Examples of heterocycloalkyl groups include tetrahydrofuryl, morpholinyl, piperazinyl, piperidyl, and pyrrolidinyl. Preferred heterocycloalkyl groups are five and six membered rings and contain from one to three heteroatoms. It is also possible for the heterocycloalkyl group to have one or more double bonds, but is not aromatic. Example of heterocycloalkyl groups containing double bonds include dihydrofuran, and the like.

The term “short cysteine containing peptide” as used herein refer to peptides containing cysteine having a total of, for example, 2-20, 2-15, 2-10, 2-5, 3-15, 3-10, 3-5 or 5-10 amino acids.

The term “zinc-finger motif” encompasses zinc-finger motif” in fungi or yeast.

It is noted that the cyclic ring groups, i.e., aryl, heteroaryl, cycloalkyl, heterocycloalkyl, can have more than one ring. For example, the naphthyl group is a fused bicyclic ring system. Anti-fungal compounds can also include ring groups that have bridging atoms, or ring groups that have a spiro orientation. Representative examples of five to six membered aromatic rings, optionally having one or two heteroatoms, are phenyl, furyl, thienyl, pyrrolyl, oxazolyl, thiazolyl, imidazolyl, pyrazolyl, isoxazolyl, isothiazolyl, pyridinyl, pyridazinyl, pyrimidinyl, and pyrazinyl.

Representative examples of partially saturated, fully saturated or fully unsaturated five to eight membered rings, optionally having one to three heteroatoms, are cyclopentyl, cyclohexyl, cycloheptyl, cyclooctyl and phenyl. Further exemplary five membered rings are furyl, thienyl, pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadiazaolyl, 1,2,4-oxadiazolyl, 1,2,5-oxadiazolyl, 1,3,4-oxadiazolyl, 1,2,3-triazolyl, 1,2,4-triazaolyl, 1,3,4-thiadiazolyl, 3H-1,2,3-dioxazolyl, 1,2,4-dioxazolyl, 1,3,2-dioxazolyl, 1,3,4-dioxazolyl, 5H-1,2,5-oxathiazolyl,and 1,3-oxathiolyl.

Further exemplary six membered rings are 2H-pyranyl, 4H-pyranyl, pyridinyl, piperidinyl, 1,2-dioxinyl, 1,3-dioxinyl,1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, and 1,4,2-oxadiazinyl. Further exemplary seven membered rings are azepinyl, oxepinyl, thiepinyl and 1,2,4-triazepinyl.

Further exemplary eight membered rings are cyclooctyl, cyclooctenyl and cyclooctadienyl.

Exemplary bicyclic rings consisting of two fused partially saturated, fully saturated or fully unsaturated five and/or six membered rings, taken independently, optionally having one to four heteroatoms are indolizinyl, indolyl, isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, anthranilyl, benzimidazolyl, benzthiazolyl, purinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2-H1-1-benzopyranyl, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

A cyclic ring group may be bonded to another group in more than one way. If no particular bonding arrangement is specified, then all possible arrangements are intended. For example, the term “pyridyl” includes 2-, 3-, or 4-pyridyl, and the term “thienyl” includes 2-, or 3-thienyl.

The term “substituted” means that a hydrogen atom on a molecule has been replaced with a different atom or with a molecule. The atom or molecule replacing the hydrogen atom is called a subsistent. Examples of suitable substituents include, halogen, —OC1-C8 alkyl, —C1-C8 alkyl, —CF₃, —NH₂, —NHC1-C8 alkyl, —N(C1-C8 alkyl)₂, —NO₂, —CN, —CO₂H, —CO₂C1-C8 alkyl, and the like.

The symbol “—” represents a covalent bond.

II. Zinc Finger Motifs and Anti-Fungal Drug Targets

The six-cysteine (Zn(II)₂Cys₆ or C₆ zinc) binuclear cluster DNA binding domain was first characterized in the Saccharomyces cerevisiae GAL4 protein. The six cysteine residues, arranged C—X₂—C—X₆—C—X₆—C—X₂—C—X₆—C, coordinate two zinc(II) ions to form a cloverleaf-shaped structure (FIG. 1 of Pan and Coleman, Proc. Natl. Acad. USA 87(6):2077-2081 (1990); Gardner et al., Biochemistry 30(47), 11292-11302 (1991). The second, third, fifth, and sixth cysteine residues act as terminal ligands, whereas the first and fourth cysteine residues act as bridging ligands by ligating both metal ions (Pan and Coleman, 1991; Gardner et al., 1991). This motif can be viewed as two C—X₂—C—X₆—C repeat units separated by six residues. Each of these units forms short α-helical structures separated by a loop with a proline-associated turn (Baleja et al., Nature 356(6368):450-453 (1992); Kraulis, et al., Nature 356(6368):448-450 (1992); Marmorstein et al., Nature(6368):408-414 (1992)). The zinc(II)-binding cluster lies in the DNA major groove and contacts three base pairs (Marmorstein et al., 1992). The Zn(II)₂Cys₆ motif of the S. cerevisiae PPRI protein coordinates two zinc(II) ions via the six cysteine residues and forms a structure nearly identical in conformation to that observed for GAL4 (Marmorstein and Harrison, Genes 8(20)2504-2512 (1994); Ball et al., FEBS Letter 358(3):278-282(1995)). Coordination of two zinc(II) ions and/or DNA binding has now been documented for a number of other Zn(II)₂Cys₆ proteins (Todd, supra, Table 1). DNA binding by these proteins occurs in a sequence-specific manner (Todd, supra, Table 2), and many have been shown to bind DNA as a dimer (Todd, supra, Table 1).

Zinc finger structural motifs typically contain conserved cysteine and histidine residues that are ligands to one or more zinc ions. The cysteine and histidine residues can be arranged in a variety of structures including, for example, Cys-Xₙ-Cys-Xₙ-His-Xₙ-His, Cys-Xₙ-Cys-Xₙ-Cys-Xₙ-Cys, Cys-Xₙ-Cys-Xₙ-Cys-Xₙ-His, Cys-Xₙ-His-Xₙ-Cys-Xₙ-Cys, His-Xₙ-Cys-Xₙ-Cys-Xₙ-Cys, Cys-Xₙ-Cys-Xₙ-Cys-Xₙ-Cys-Xₙ-Cy-Xₙ-Cys (where X is any amino acid). Zinc fingers in proteins have a variety of functions in RNA or DNA binding and protein-protein interactions. Many of these proteins are transcription factors that function by recognition of specific DNA or RNA sequences and are often essential for the cellular viability (i.e. the transcription factor TFIIIA).

The binding specificity and affinity of a zinc finger is largely determined to a large degree by the amino acid residues which contact the nucleic acids of the polynucleotide. Based on the published reports of the x-ray crystal structures of zinc fingers complexed to DNA, there are four nucleic acid-contacting residues in zinc fingers that are primarily responsible for determining specificity and affinity. These four amino acid residues occur in the same position relative to the first consensus histidine and the second consensus cysteine. Specifically, these four amino acid residues define which three to four base pair or subsites the zinc finger prefers to bind (i.e. the specificity of the zinc finger) and with how great an affinity. The first of the three critical amino acid residues is seven residues to the N-terminal side of the first consensus histidine and six residues to the C-terminal side of the second consensus cysteine. The other three amino acids are two, three and six residues removed from the C-terminus of the residue at position. The amino acid residues one and five residues removed from the C-terminus of the amino acid at the −1 position are also important to zinc finger specificity and binding strength.

Other zinc finger-containing proteins have critical roles in mediating protein-protein interactions (i.e. the eukaroytic protein GATA-1 binding to the FOG family of proteins) and in RNA recognition and binding (i.e. the NCp7 protein in the human immunodeficiency virus). A number of applications for zinc finger technology have been suggested, including the treatment of diseases, use as reagents for manipulating nucleic acids and the regulation of gene expression. In particular, the NCp7 zinc finger protein of HIV is the model system in which the chemical selectivity of specific small molecules has been principally developed. The NCp7 and other zinc finger sequences recognize specific RNA (and in some cases DNA) motifs. RNA motif is a term generally used to describe the secondary or tertiary structure of RNA molecules. The primary sequence of an RNA is a specific string of nucleotides (A, C, G or U) in one dimension. The primary sequence does not give information on first impression as to the three dimensional configuration of the RNA, although it is the primary sequence that dictates the three-dimensional configuration.

The secondary structure of an RNA motif is represented by contact in two dimensions between specific nucleotides. The most easily recognized secondary structural motifs are comprised of the Watson/Crick basepairs A:U and C:G. Non-Watson/Crick basepairs, often of lower stability, have been recognized, and include the pairs G:U, A:C, G:A, and U:U.

The conventional nomenclature for the RNA secondary structures includes hairpin loops, asymmetric bulged hairpin loops, symmetric hairpin loops, and pseudoknots. When nucleotides that are distant in the primary sequence and not thought to interact through Watson/Crick and non-Watson/Crick base pairs are in fact interacting, these interactions (which are often depicted in two dimensions) are also part of the secondary structure.

Because zinc finger proteins often have essential cellular functions, it is possible to develop drugs targeted to zinc finger-containing proteins in fungi or yeast. For example, the Zn(II)₂Cys₆ binuclear zinc cluster (“binuclear cluster”) is a zinc finger structural domain with the consensus sequence Cys-X₂-Cys-X₆-Cys-X₆-Cys-X₂-Cy-X₆-Cys that has been identified in fungi, but not in mammals, insects, bacteria or other types of organisms. The binuclear cluster is thus uniquely found in fungi but not in human cells and thereby provides a target to develop drugs targeted to the fungal cluster that will not interfere with human cellular function. The binuclear cluster is a Cys₆-Zn(II)₂ type zinc finger domain that utilizes six cysteine sulfur atoms to coordinate two zinc atoms into a fungal-specific defined structure. Sequence conservation analysis has determined that the six cysteine residues are 100% conserved among all known fungal binuclear cluster proteins. Thus, the binuclear cluster represents a highly conserved domain that may not permit mutational alterations. Drugs targeted against fungal binuclear clusters may thus be less prone to promote the generation of resistant fungal strains.

Zinc finger domains provide certain proteins with the ability to interact selectively with specific sequences of nucleic acids or with other protein sequences. For example, the fungal binuclear cluster is known to endow particular fungal proteins with specific nucleic acid sequence recognition capabilities. The potential zinc finger protein targets of importance to the Candida are represented below in Table 1.

TABLE 1
Important Zinc Finger Protein Targets in Candida.

| Name | Function | Type |
|---|---|---|
| SUC1 | sucrose utilization | Zn(2)-Cys(6) |
| ZF1 | cell cycle transductionZn(2)-Cys(6) | |
| POL III | DNA polymerase | |
| ROK1 | helicase | |
| SIR1 | transcription | CCHCC |
| CAA21953 | DNA repair | C(3)HC(4) |
| CAA21933 | DNA repair | Zn(2)-Cys(6) |
| NRG1 | transcription repressor | |
| ZNF1 | cell cycle arrest | Cys(2)HHHCys(2) |

The zinc finger motif in proteins was first identified in the Xenopus transcription factor TFIIIA. Since then over 200 proteins have been identified that contain zinc finger motifs with some proteins containing more than one finger. Zinc fingers in proteins have been demonstrated to be involved in a variety of cellular functions, including binding to DNA, binding to RNA, protein-protein interactions, and lipid binding. These motifs are characterized by a defined set of cysteine or histidine residues or a mixture of these two residues arranged in characteristic motifs. The motifs include Cys-Xₙ-Cys-Xₙ-His-Xₙ-His, Cys-Xₙ-Cys-Xₙ-Cys-Xₙ-Cys, Cys-Xₙ-Cys-Xₙ-Cys-Xₙ-His, Cys-Xₙ-His-Xₙ-Cys-Xₙ-Cys, His-Xₙ-Cys-Xₙ-Cys-Xₙ-Cys, Cys-Xₙ-Cys-Xₙ-Cys-Xₙ-Cys-Xₙ-Cy-Xₙ-Cys (where X is any amino acid). In each of these motifs zinc ions are coordinated by either the sulfhydryls of the cysteine residues or one of the heterocyclic nitrogens of histidine. This coordination results in a defined structure that presents amino acids that either flank the motif or lie between the ligating residues in specific architectures that are required the function of the protein. In the majority of cases, mutation or modification of the zinc finger motif such that it no longer forms a stable complex with zinc results in the loss of the function of the protein.

III. Methods Screening for Agents that Selectively Bind to Fungal or Yeast Zinc Finger-Containing Proteins

In general, the methods described herein involve: (i) adding a test compound to a culture medium of fungal or yeast cells that express zinc finger proteins, (ii) incubating the fungal or yeast cells for a period of, for example, hours, days, weeks, or months, and (iii) measuring the viability of the fungal or yeast cells.

Fungicidal or yeast-cidal compounds may act though mechanisms other than targeting the zinc-finger motif within the fungi or yeasts. To differentiate anti-fungal or anti-yeast compounds that target fungal zinc finger proteins from compounds which produce antifungal actions by other mechanisms, antifungal assays are performed preferably in the presence and in the absence of compounds that block the action of zinc finger-selective fungicidal or yeast-cidal agents. The blocking compounds provide a comparable environment to the zinc-finger motif within the fungi or yeasts. A higher concentration of the blocking compounds then provides a higher total concentration of the zinc-finger motif and the blocking compounds, therefore providing a strong competing force in thermodynamics against the test compound.

Some fungal strains or yeast strains may be inhibited by the blocking compound alone, and other fungal strains are known to be growth-stimulated by high concentrations of some of the blocking compounds, such as thioacetic acid, which is a normal constituent of certain microbiological media. Accordingly, a blocking compound which does not cause this stimulation or inhibition of the particular fungal strain under study may be used. A blank control test including only the blocking compound can be performed to ensure that the blocking compound does not inhibit or substantially inhibit fungi or yeast cells.

In order to demonstrate selective zinc finger antifungal activity, growth can be assessed in the presence and the absence of a blocking compound. The test compound is designated as effective if the test compound inhibits in the absence of the blocking compound but the antifungal activity of the test compound is greatly inhibited or totally abolished in the presence of a high concentration of the blocking compound. Further, In order to determine whether a test compound is effective in inhibiting fungal or yeast cells at a certain concentration, it may be necessary to vary the concentration of the test compound in the assay process. The concentration of the test compound can vary from, for example, about 0.01 nM to 50 μM.

The viability of the fungal or yeast cells can be determined by any method available in the art.

The efficacy of the test compound against the fungal or yeast cells may also be determined by measuring the zinc ejection resulting from the reaction of the test compound and the zinc finger protein using a zinc assay, for example zinc fluorescence assay. The measurements of the zinc ejection level can be compared against a zinc level standard available in the art or a standard zinc level curve to determine the inhibitory effect of the test compound and the dosage of the compound against a particular type of fungal or yeast cells.

In one embodiment, the method for screening for compounds which bind to or associate with fungal zinc finger motif includes the following steps: (i) adding an active sulfhydryl compound to a first plurality of fungal or yeast cells which express zinc finger proteins, (ii) subsequently adding the test compound, (iii) incubating the fungal or yeast cells in the presence of the sulfhydryl compound and the test compound, and (iv) taking a first measurement of the viability of the fungal or yeast cells. The method may further include: (v) adding a test compound to a second plurality of fungal or yeast cells which express zinc finger proteins, (vi) incubating fungal or yeast cells in the presence of the test compound, (vii) taking a second measurement of the viability of the fungal or yeast cells, and (viii) comparing the first and the second measurement of the viability of the fungal or yeast cells to designate the test compound as effective or non-effective for binding to or associating with fungal zinc finger motif.

In a further embodiment, the method for screening for compounds which bind to or associate with fungal zinc finger motif includes the steps of: (i) adding an active sulfhydryl compound to a first plurality of fungal or yeast cells which express zinc finger proteins, (ii) subsequently adding the test compound, (iii) incubating the fungal or yeast cells in the presence of the test compound and the active sulfhydryl compound, and (iv) taking a first measurement of the zinc ejection resulted from the reaction of the compound and the zinc finger by a zinc florescence assay. The method may further include: (v) adding a test compound to a second plurality of fungal or yeast cells which express zinc finger proteins, (vi) incubating fungal or yeast cells with the test compound, (vii) taking a second measurement of the zinc ejection resulted from the reaction of the compound and the zinc finger by a zinc florescence assay, and (viii) comparing the first and the second measurements of the zinc ejection to designate the test compound as effective or non-effective for binding to or associating with fungal zinc finger motif.

A. Agents that Selectively Bind to Fungal or Yeast Zinc Finger-Containing Proteins

Any compound can be tested for selective zinc finger antifungal activity. Since the antifungal agents bind to and alter the structure of the zinc finger motif they may cause zinc release from fungal zinc finger-containing proteins. The antifungal agents may have one or more groups that form a coordination bond with one or more Zn(II) ions. The groups that coordinate to Zn(II) ions generally have one or multiple nitrogen, sulfur, halogen, oxygen, phosphorous, or carbon atoms. One example of such a group is an amino group. Another example is a sulfide or disulfide group. Still anther example is the imidazole group. One skilled in the art can determine which group can coordinate to a Zn(II) atom. The antifungal agent may have redox properties that can lead to the selective oxidation of one or more of the cysteine residues of the binuclear zinc cluster. One example is antifungal agents bearing one or more nitroso groups.

The zinc ejection by the anti-fungal agents can have various mechanisms. In one representative mechanism, the antifungal agent can selectively oxidize one of the cysteine residues of the zinc finger to eject the zinc ion. In another representative mechanism, the anti-fungal agent can selectively chelate to the zinc ion of the zinc finger. Representative anti-viral compounds operating by this mechanism have been described in the literature, for example, C-nitroso compounds described by Rice et al., Nature, 361:473-475 (1993); 3-nitrosobenzamide (NOBA) and its derivatives, disulfide-substituted benzamides and their derivatives described by Rice et al., Science, 270:1194-97 (1995); azodicarbonamide compounds and their derivatives as described by Rice et al., Nat. Med. 3:341-345 (1997); dithiane compounds and their derivatives as described by Antimicrobial. Agents Chem., 41:419-26 (1997); compounds described in references 21-27 in Huang et al., J. Med. Chem., 41:1371-1381 (1998); zinc chelators as described by Otsuka et al., J. Med. Chem. 38:3264-3270 (1995); and disulfide benzamides and benzisothiazolones described by Tummino et al., Antimicrob. Agents Chem., 41:394-400. The compounds and the methods of preparation therefore described in each reference and all references within each of the references are incorporated herein by reference.

In one embodiment, the zinc ejecting molecules have one of the structures:

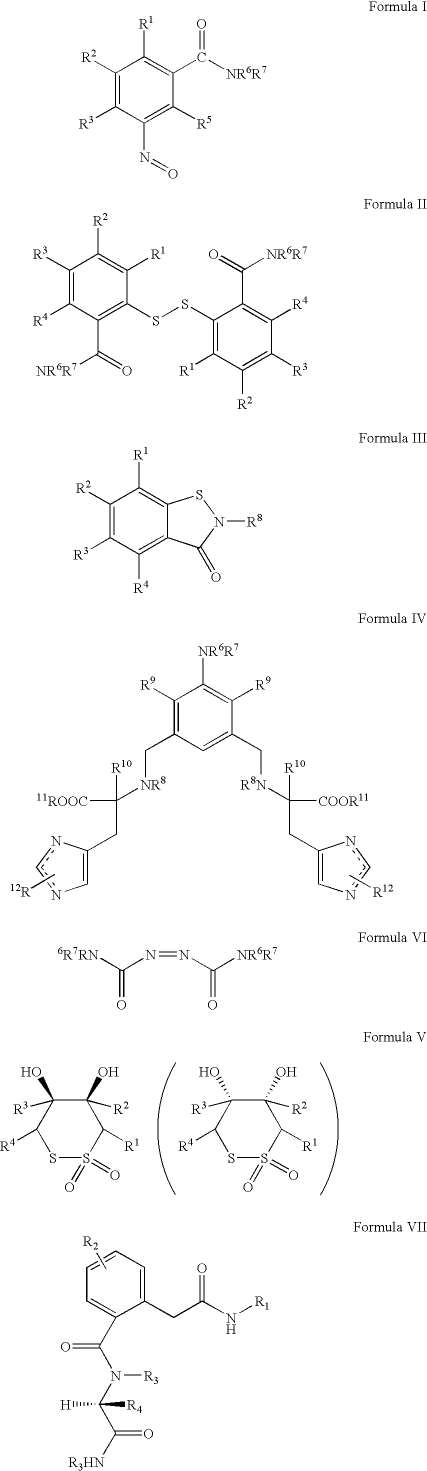

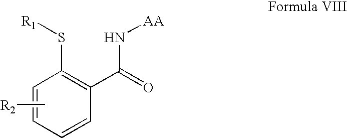

wherein R¹, R², R³ and R⁴ independently are H, halo, C1-C10 alkyl, C1-C10 alkenyl, C1-C10 alkynyl, C1-C10 phenyl, C1-C10 sulfonamidophenyl, C1-C10 aryl, arylalkyl, C1-C10 cycloalkyl, C1-C10 carbyl carbonyl, C1-C10 carboxy, C1-C10 carboxy, C1-C10 amido, C1-C10 amino, C1-C10 oxy, C1-C10 sulfido, acyl which may optionally contain alkyl, cycloalkyl, alkenyl, amide, alkyl amide, or aryl amide, heterocyclic, thioalkyl, thioalkyl optionally substituted at an alkyl position with an aryl or heterocyclic group, amino acid, peptide, or polypeptide;

wherein R⁵ and R⁹ independently are H, halo, CH₃, ethyl, ethenyl, or ethynyl;

wherein R⁶, R⁷, and R⁸ are independently H, C1-C3 alkyl, C1-C3 alkenyl, C1-C3 alkynyl or cyclopropanyl;

wherein R¹¹ is H, C1-C6 alkyl, C1-C6 alkenyl, C1-C6 alkynyl, C1-C6 cycloalkyl or C1-C6 phenyl;

wherein R¹² is H, C1-C6 alkyl, C1-C6 alkenyl, C1-C6 alkynyl, C1-C6 cycloalkyl, C1-C6 alkylphenyl or triphenylmethyl; and

wherein AA is an amino acid group or peptide group.

Representative compounds have the structure of Formula I wherein R¹, R², and R³ are low alkyl groups and wherein R⁵, R⁶ or R⁷ is H. One preferred compound has a structure of Formula I wherein R⁵, R⁶ and R⁷ are H. One of the most preferred compounds has a structure of Formula I which is 3-nitrosobenamine (NOBA).

Other representative compounds have the structure of Formula II wherein R¹, R², R³ and R⁴ are lower alkyl groups. One preferred compound has a structure of Formula II wherein R⁶ and R⁷ are H. One of the most preferred compounds are wherein R¹, R², R₃,R⁴, R⁶ and R⁷ are H groups.

Still other representative compounds have a structure of Formula III wherein R¹, R², R³ and R⁴ are low alkyl groups. One preferred compound has a structure of Formula III wherein R⁸ is H. One most preferred compound has a structure of Formula III wherein R¹, R², R³,R⁴ and R⁸ are H groups.

Another representative compound has a structure of Formula IV wherein R⁶, R⁷, R⁸, R¹⁰ and R¹¹ are lower alkyl groups. Another preferred compound has a structure of Formula IV wherein R¹² is either H, CH₃ or triphenylmethyl. The most preferred compound is wherein R⁶, R⁷, R⁸, R¹⁰ and R¹¹ are H groups.

Still another representative compound has a structure of Formula V wherein R¹, R², R³ and R⁴ are lower alkyl groups. One preferred compound has a structure of Formula V wherein R¹, R², R³ and R⁴ are H groups. Another preferred compound has a structure of Formula VI wherein R⁶ or R⁷is a lower alkyl or H group. One of the most preferred compounds has a structure of Formula VI wherein R⁶ and R⁶ are H groups.

Other preferred compounds correspond to the structure of Formula VII wherein R¹ is acyl, with the acyl group containing an alkyl, cycloalkyl, or alkenyl group; R² is hydrogen, halogen, alkoxy, fluoroalkyl, or fluoroalkoxy; and R³ is represented by any of the 22 common naturally occurring amino acids, in either the L- or D-conformation coupled via its amino group. In these compounds the amino acid acyl group may be substituted by an amide or alkyl amide or aryl amide.

Other preferred compounds correspond to the structure of Formula VII or Formula VIII wherein R¹ is alkyl or arylalkyl or heterocyclic or independently thioalkyl or thioalkyl optionally substituted at an alkyl position from C1 to C3 alkyl with an aryl or heterocyclic group and wherein AA represents one of the 22 common amino acids coupled via its amino group. In these compounds the amino acid acyl group may be substituted by an amide or alkyl amide or arylalkyl amide. The amino acid residue may be in the naturally occurring L or the unnatural D conformation.

Antifungal compounds are either commercially available or can be readily synthesized by one normally skilled in the art. The synthesis of compounds of Formula I-VI can be readily carried out using the knowledge in the art of organic synthesis or are otherwise commercially available. The above references which are specifically incorporated herein fully describe the preparation of useful compounds. For example, while NOBA is commercially available, derivatives of NOBA of Formula I can be prepared following the methods available in the art. Benzisothiazolones can be synthesized by, for example, the cyclization method described in Tummino et al., supra. The synthesis of compounds Formula IV is fully described, for example, in Otsuka et al., supra.

B. Compounds that Block the Action of Zinc Finger-Selective Fungicidal or Yeast-Cidal Agents

Preferably, sulfhydryl compounds are used as blocker compounds to differentiate compounds that inhibit fungi or yeast by binding to or associating with the zinc-finger motif within the fungi or yeast. The concentration of the active sulfhydryl compound is typically at a higher concentration than is used for the test compound, for example, about 5, 10, 20, 50, 100, 200, 500, or 1,000 times of the test compound, ranging from about 1 μM-500 μM, 10 μM-500 μM, 50 μM-500 μM, 80 μM-500 μM, or 100 μM-300 μM, typically about 100 μM.

Representative blocking compounds include, but are not limited to, Cysteine, glutathione (reduced), glu-cys-gly, glu-cys-glu, glu cys-gly, glu-cys-cys-glu, glu-cys-pro-arg, glu-cys-arg, glu-cys-gln, gln-cys-gln, gln-cys-asn, asn-cys-asn, asn-cys-asn-cys-asn, asn-cys-pro-cys-asn, asn-cys-gly-cys-gln, asn-cys-gly-cys-gly-gln, cys-cys-cys, cys-cys, cys-met, cys-met-asn, asn-cys-met-asn, gln-cys-met-asn, cys-gly-pro-gly-cys-gly-pro-gly, asn-cys-gly-pro-gly-cys-gly-pro-gly-asn, gln-cys-gly-pro-gly-cys-gly-pro-gly-gln, glu-cys-gln-cys-glu, asp-cys-gln-cys-asp, gln-cys-val-met-phe-cys-gln, gln-cys-ile-met-phe-cys-gln, gln-cys-phe-met-phe-cys-gln, cys-cys-cys-cys, gln-cys-cys-cys-cys-gln, gln-cys-cys-cys-cys-asn, asn-cys-cys-cys-cys-asn, glu-cys-cys-cys-cys-gln, gln-cys-cys-gln-cys-cys-gln,gamma-carboxy Glu-cys, gamma-carboxy Glu-cys-asn, gamma-carboxy Glu-cys-gln, gamma-carboxy Glu-cys-cys, gamma-carboxy Glu-cys-cys-asn, 2-mercaptobenzimidazole, mercaptoacetic acid, thiosalicylic acid, 3-mercaptobenzoic acid, 4-mercaptobenzoic acid, 2-mercaptobenzothiazole, 3-mercapto-2-butanol, 4-mercapto-1-butanol, 2-mercaptoethanesulfonic acid, 2-mercaptoethanol, 2-mercaptoethyl sulfide, 2-mercaptoethyltrimethylammonium halides, 6-merapto-1-hexanol,2-mercaptoimidazole, 2-mercapto-5-methylbenzimidazole, 2-mercapto-5-methyl benzothiazole, 2-mercapto-5-methoxy benzothiazole, 2-mercapto-5-methoxybenzothiazole, 2-mercapto-5-chlorobenzothiazole, 2-mercapto-5-chlorobenzimidazole, 2-mercapto-1-methylimidazole, 3-mercaptopropionic acid, thiolactic acid, 3-mercapto-1-propanol, 1-mercapto-2-propanol, 3-mercapto-1-propanesulfonic acid, 3-mercapto-1,2-propanediol, 2-mercapto-5-amino-benzimidazole, 2-mercapto-5-amino-benzothiazole, 2-mercaptonicotinic acid, 6-mercaptonicotinic acid, captopril, 5-mercapto-1H-tetrazole, mercaptosuccinic acid, mercaptopyruvic acid, 2-mercaptopyridine, 2-mercaptopyridine-1-oxide, 4-mercaptopyridine, 3-mercaptopyridine, 6-mercaptopurine, 2-mercaptothiazoline, 2-mercaptothiazoline-4-carboxylic acid, thiobenzoic acid, thiooctic acid (reduced form), thiolactic acid. Useful blocking compounds also include methyl, ethyl, and isopropyl esters, amides, and N-methylamides of the acids disclosed herein.

IV. Formulations Containing Compounds that Selectively Bind to Fungal or Yeast Zinc-Finger Proteins

The compounds identified by the methods described herein that bind to fungal or yeast zinc-finger proteins can be formulated into pharmaceutical, agricultural and industrial formulations. The formulations generally contains an effective amount of a compound or compounds identified as effective for binding to fungal or yeast zinc-finger proteins using the screening methods described herein. The formulations may include any pharmaceutically acceptable carrier for enteral or parenteral administration, excipients, and/or polymeric materials for immediate and/or sustained release formulations as described in U.S. application Ser. No. 10/260,505.

The formulations can be used for treating fungal and/or yeast infections by administering to a patient in need of treatment a composition containing an effective amount of a compound or compounds identified as effective for binding to fungal or yeast zinc-finger proteins using the screen methods described herein.

The methods and compositions disclosed herein can be further understood by the following non-limiting examples.

### EXAMPLES

### Example 1

Determination of Fungal Susceptibility to Zinc Finger Selective Antifungals

A panel of Candida clinical isolates (including azole-resistant species) plus single isolates of Aspergillus fumigates, were used to evaluate the activity of the test compounds in vitro. Inocula were prepared as broth cultures (yeasts) or as suspensions of fungal material made from agar slope cultures (molds). The test compounds were pipetted from DMSO stock solution into water to provide a series of 10-fold dilutions. The fungal inocula were suspended in the growth medium CYG (F. C. Odds, J. Clin. Microbiol. 29, 2735-2740 (1991) at approximately 50000 colony-forming units (CFU) per ml and added to the aqueous test drugs. The test medium alone did not contain glutathione, cysteine, or other readily small molecules which would block the action of zinc finger reactive small molecules. Alternate wells contained 10 mM cysteine in growth medium as the zinc finger blocking compound. The cultures were set up in the 96 wells of plastic microdilution plates and they were incubated for 2 days at 37° C. (Candida spp.) or for 5 days at 30° C. (other fungi). Growth in the microcultures was measured by its optical density (OD) measured at a wavelength of 405 nm. The OD for cultures with test compounds was calculated as a percentage of the control, drug-free OD. Inhibition of growth to 35% of control or less was recorded as significant inhibition (Table 2). It had previously been determined that the cysteine (or other zinc finger blocking compounds which were evaluated) did not enhance or retard fungal growth or proliferation when applied alone.

Similar results were observed for Aspergillus cultures and additional fungal strains that were examined.

TABLE 2
Minimum Inhibition Concentration of compounds against
Candida

| | | C. albicans | C. albicans | |
|---|---|---|---|---|
| | C. albicans | MIC + 200 | MIC + | |
| | MIC | μg/ml | 200 μg/ml | Fold |
| Compound | (μg/ml) | cysteine | glutathione | Increase |
| 2 | 4 | 16 | 16 | 4 |
| 4 | 16 | 16 | 16 | 0 |
| 6 | 8 | 16 | 16 | 2 |
| 9 | 0.25 | 8 | 1 | 4-32 |
| 12 | 2 | 16 | 16 | 8 |

| | |
|---|---|
| 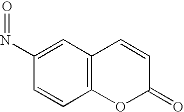 2 6-nitroso-1,3-benzopyrene | 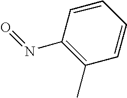 4 2-nitrosotoluene |
| 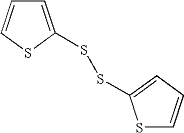 6 di(2-thienyl)disulphide | 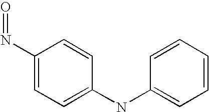 9 4-nitroso-N,N-dimethylaniline |
| 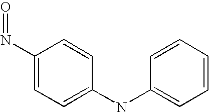 12 4-nitrosodiohenylamine | |

Discussion

The data of Table 2 indicate that two blocking agents glutathione and cysteine are roughly equivalent at the concentrations at which they were employed in this study. The only exception to this, 4-nitroso-N,N-dimethylaniline, is blocked to a somewhat different extent by the two different blocking compounds, but in both cases the effect is significant.

One of the compounds, 2-nitrosotoluene, does not appear to exert its fungicidal action through a zinc finger mechanism, as it is not blocked by either glutathione or cysteine, unlike the other closely related compounds in Table 2. This result indicates the utility of the present approach in elucidating the mechanistic basis of antifungal action of compounds which can react with zinc finger motifs in functionally important proteins.

Publications and references cited herein and the material for which they are cited are specifically incorporated by reference. Modifications and variations of the present invention will be obvious to those skilled in the art from the foregoing detailed description and are intended to be encompassed by the following claims.

We claim:

1. A method for screening for compounds which bind to or associate with a fungal zinc finger motif, comprising
(i) adding an active sulfhydryl compound to a first plurality of fungal or yeast cells which express zinc finger proteins,
(ii) subsequently adding the test compound,
(iii) incubating the fungal or yeast cells in the presence of the sulfhydryl compound and the test compound, and
(iv) taking a first measurement of the viability of the fungal or yeast cells.

2. The method of claim 1, further comprising
(v) adding a test compound to a second plurality of fungal or yeast cells which express zinc finger proteins,
(vi) incubating fungal or yeast cells in the presence of the test compound,
(vii) taking a second measurement of the viability of the fungal or yeast cells, and
(viii) comparing the first and the second measurement of the viability of the fungal or yeast cells to designate the test compound as effective or non-effective for binding to or associating with fungal zinc finger motif,
wherein the active sulfhydryl compound is added at a higher concentration than is used for the test compound.

3. The method of claim 1 wherein the test compound is added at a concentration from about 0.01 nM to about 50 μM.

4. The method of claim 3 where the active sulfhydryl compound is cysteine.

5. The method of claim 3 where the active sulthydryl compound is reduced glutathione.

6. The method of claim 3 where the active sulfhydryl compound is a short cysteine containing peptide which is sufficiently hydrophilic to allow access of the cysteine sulfhydryl to exposed zinc finger sites.

7. The method of claim 3 where the active sulfydryl compound is selected from the group consisting of 2-mercaptobenzimidazole, 2-mercaptobenzothiazole, 3-mercapto-2-butanol, 4-mercapto-1-butanol, 2-mercaptoethanesulfonic acid, 2-mercaptoethanol, 2-mercaptoethyl sulfide, 2-mercaptoethyltrimethylammonium halides, 6-merapto-1-hexanol,2-mercaptoimidazole, 2-mercapto-5-methylbenzimidazole, 2-mercapto-5-methyl benzothiazole, 2-mercapto-5-methoxy benzothiazole, 2-mercapto-5-methoxybenzothiazole, 2-mercapto-5-chlorobenzothiazole, 2-mercapto-5-chlorobenzimidazole, 2-mercapto-1-methylimidazole, 3-mercaptopropionic acid, thiolactic acid, 3-mercapto-1-propanol, 1-mercapto-2-propanol, 3-mercapto-1-propanesulfonic acid, 3-mercapto-1,2-propanediol, 2-mercapto-5-amino-benzimidazole, 2-mercapto-5-amino-benzothiazole captopril, 5-mercapto-1H-tetrazole, 2-mercaptopyridine, 2-mercaptopyridine-1-oxide, 4-mercaptopyridine, 3-mercaptopyridine, 6-mercaptopurine, and 2-mercaptothiazoline.

8. The method of claim 3 wherein the active sulthydryl compound is selected from the group consisting of mercaptoacetic acid, thiosalicylic acid, 3-mercaptobenzoic acid, 4-mercaptobenzoic acid, 3-mercaptopropionic acid, thiolactic acid, 2-mercaptonicotinic acid, 6-mercaptonicotinic acid, mercaptosuccinic acid, mercaptopyruvic acid, 2-mercaptothiazoline-4-carboxylic acid, thiobenzoic acid, thiooctic acid (reduced form), thiolactic acid, and salts of the acids.

9. The method of claim 3 wherein the active sulfhydryl compound is an amide, N-methyl amide, N-isopropyl amide, methyl ester, ethyl ester, or n-propyl ester of an acid selected from the group consisting of mercaptoacetic acid, thiosalicylic acid, 3-mercaptobenzoic acid, 4-mercaptobenzoic acid, 3-mercaptopropionic acid, thiolactic acid, 2-mercaptonicotinic acid, 6-mercaptonicotinic acid mercaptosuccinic acid, mercaptopyruvic acid, 2-mercaptothiazoline-4-carboxylic acid, thiobenzoic acid, thiooctic acid (reduced form), and thiolactic acid.

10. A method for screening for compounds which bind to or associate with a fungal zinc finger motif, comprising
(i) adding an active sulfhydryl compound to a first plurality of fungal or yeast cells which express zinc finger proteins,
(ii) subsequently adding the test compound,
(iii) incubating the fungal or yeast cells in the presence of the test compound and the active sulfhydryl compound, and
(iv) taking a first measurement of the zinc ejection resulted from the reaction of the compound and the zinc finger by a zinc florescence assay.

11. The method of claim 10, further comprising
(v) adding a test compound to a second plurality of fungal or yeast cells which express zinc finger proteins,
(vi) incubating fungal or yeast cells with the test compound,
(vii) taking a second measurement of the zinc ejection resulted from the reaction of the compound and the zinc finger by a zinc florescence assay, and
(viii) comparing the first and the second measurements of the zinc ejection to designate the test compound as effective or non-effective for binding to or associating with fungal zinc finger motif,
wherein the active sulfhydryl compound is added at a higher concentration than is used for the test compound.

12. The method of claim 10 wherein the test compound is added at a concentration varying from about 0.01 nM to about 50 uM.

13. The method of claim 12 further comprising determining a level of zinc ejection by measuring the zinc florescence against a standard zinc level curve.

14. The method of claim 12 where at least one of the steps, is carried out by automation.

15. The method of claim 12 where the active sulfhydryl compound is cysteine.

16. The method of claim 12 where the active sulfhydryl compound is reduced glutathione.

17. The method of claim 12 where the active sulfhydryl compound is a short cysteine containing peptide which is sufficiently hydrophilic to allow access of the cysteine sulthydryl to exposed zinc finger sites.

18. The method of claim 12 where the active sulfhydryl compound is selected from the group consisting of 2-mercaptobenzimidazole, 2-mercaptobenzothiazole, 3-mercapto-2-butanol, 4-mercapto-1-butanol, 2-mercaptoethanesulfonic acid, 2-mercaptoethanol, 2-mercaptoethyl sulfide, 2-mercaptoethyltrimethylammonium halides, 6-merapto-1-hexanol,2-mercaptoimidazole, 2-mercapto-5-methylbenzimidazole, 2-mercapto-5-methyl benzothiazole, 2-mercapto-5-methoxy benzothiazole, 2-mercapto-5-methoxybenzothiazole, 2-mercapto-5-chlorobenzothiazole, 2-mercapto-5-chlorobenzimidazole, 2-mercapto-1-methylimidazole, 3-mercaptopropionic acid, thiolactic acid, 3-mercapto-1-propanol, 1-mercapto-2-propanol, 3-mercapto-1-propanesulfonic acid, 3-mercapto-1,2-propanediol, 2-mercapto-5-amino-benzimidazole, 2-mercapto-5-amino-benzothiazole captopril, 5-mercapto-1H-tetrazole, 2-mercaptopyridine, 2-mercaptopyridine-1-oxide, 4-mercaptopyridine, 3-mercaptopyridine, 6-mercaptopurine, and 2-mercaptothiazoline.

19. The method of claim 12 wherein the active sulfydryl compound is selected from the group consisting of mercaptoacetic acid, thiosalicylic acid, 3-mercaptobenzoic acid, 4-mercaptobenzoic acid, 3-mercaptopropionic acid, thiolactic acid, 2-mercaptonicotinic acid, 6-mercaptonicotinic acid, mercaptosuccinic acid, mercaptopyruvic acid, 2-mercaptothiazoline-4-carboxylic acid, thiobenzoic acid, thiooctic acid (reduced form), thiolactic acid, and salts of the acids.

20. The method of claim 12 wherein the active sulfhydryl compound is an amide, N-methyl amide, N-isopropyl amide, methyl ester, ethyl ester, or n-propyl ester of an acid selected from the group consisting of mercaptoacetic acid, thiosalicylic acid, 3-mercaptobenzoic acid, 4-mercaptobenzoic acid, 3-mercaptopropionic acid, thiolactic acid, 2-mercaptonicotinic acid, 6-mercaptonicotinic acid mercaptosuccinic acid, mercaptopyruvic acid, 2-mercaptothiazoline-4-carboxylic acid, thiobenzoic acid, thiooctic acid (reduced form), and thiolactic acid.

21. The method of claim 3 wherein the active sulfhydryl compound is used at a concentration of 100 μM.

22. The method of claim 12 wherein the active sulfhydryl compound is used at a concentration of 100 μM.

23. The method of claim 3 wherein the short cysteine containing peptide is selected from the group consisting of glu-cys-gly, glu-cys-glu, glu cys-gly, glu-cys-cys-glu, glu-cys-pro-arg, glu-cys-arg, glu-cys-gln, gln-cys-gln, gln-cys-asn, asn-cys-asn, asn-cys-asn-cys-asn, asn-cys-pro-cys-asn, asn-cys-gly-cys-gln, asn-cys-gly-cys-gly-gln, cys-cys-cys, cys-cys, cys-met, cys-met-asn, asn-cys-met-asn, gln-cys-met-asn, cys-gly-pro-gly-cys-gly-pro-gly, asn-cys-gly-pro-gly-cys-gly-pro-gly-asn, gln-cys-gly-pro-gly-cys-gly-pro-gly-gln, glu-cys-gln-cys-glu, asp-cys-gln-cys-asp, gln-cys-val-met-phe-cys-gln, gln-cys-ile-met-phe-cys-gln, gln-cys-phe-met-phe-cys-gln, cys-cys-cys-cys, gln-cys-cys-cys-cys-gln, gln-cys-cys-cys-cys-asn, asn-cys-cys-cys-cys-asn, glu-cys-cys-cys-cys-gln, gln-cys-cys-gln-cys-cys-gln, gamma-carboxy Glu-cys, gamma-carboxy Glu-cys-asn, gamma-carboxy Glu-cys-gln, gamma-carboxy Glu-cys-cys, and gamma-carboxy Glu-cys-cys-asn.

24. The method of claim 17 wherein the short cysteine containing peptide is selected from the group consisting of glu-cys-gly, glu-cys-glu, glu cys-gly, glu-cys-cys-glu, glu-cys-pro-arg, glu-cys-arg, glu-cys-gln, gln-cys-gln, gln-cys-asn, asn-cys-asn, asn-cys-asn-cys-asn, asn-cys-pro-cys-asn, asn-cys-gly-cys-gln, asn-cys-gly-cys-gly-gln, cys-cys-cys, cys-cys, cys-met, cys-met-asn, asn-cys-met-asn, gln-cys-met-asn, cys-gly-pro-gly-cys-gly-pro-gly, asn-cys-gly-pro-gly-cys-gly-pro-gly-asn, gln-cys-gly-pro-gly-cys-gly-pro-gly-gln, glu-cys-gln-cys-glu, asp-cys-gln-cys-asp, gln-cys-val-met-phe-cys-gln, gln-cys-ile-met-phe-cys-gln, gln-cys-phe-met-phe-cys-gln, cys-cys-cys-cys, gln-cys-cys-cys-cys-gln, gln-cys-cys-cys-cys-asn, asn-cys-cys-cys-cys-asn, glu-cys-cys-cys-cys-gln, gln-cys-cys-gln-cys-cys-gln, gamma-carboxy Glu-cys, gamma-carboxy Glu-cys-asn, gamma-carboxy Glu-cys-gln, gamma-carboxy Glu-cys-cys, and gamma-carboxy Glu-cys-cys-asn.

25. A method of determining an effective dosage of a compound for treating or preventing a disorder caused by a fungi or a yeast comprising
reacting a fungal or zinc finger-containing protein with the compound;
measuring the zinc ejection resulted from the reaction of the compound and the zinc finger by a zinc florescence assay;
determining a level of zinc ejection by measuring the zinc florescence against a standard zinc level curve; and
determining the effective dosage on the basis of the level of zinc ejection of the compound.

26. A pharmaceutical composition comprising an effective amount of a compound effective for binding to or associating with fungal zinc finger motif in a method for screening for the compound comprising
(i) adding an active sulfhydryl compound to a first plurality of fungal or yeast cells which express zinc finger proteins,
(ii) subsequently adding the test compound,
(iii) incubating the fungal or yeast cells in the presence of the sulfydryl compound and the test compound, and
(iv) taking a first measurement of the viability of the fungal or yeast cells.

27. The pharmaceutical composition of claim 26 wherein the method further comprising
(v) adding the test compound to a second plurality of fungal or yeast cells which express zinc finger proteins,
(vi) incubating fungal or yeast cells in the presence of the test compound,
(vii) taking a second measurement of the viability of the fungal or yeast cells, and
(viii) comparing the first and the second measurement of the viability of the fungal or yeast cells to designate the test compound as effective or non-effective for binding to or associating with fungal zinc finger motif,
wherein the active sulfhydryl compound is added at a higher concentration than is used for the test compound.

28. The pharmaceutical composition of claim 26 wherein the test compound is added at a concentration from about 0.01 nM to about 50 μM.

29. A pharmaceutical composition comprising an effective amount of a compound effective for binding to or associating with fungal zinc finger motif in a method for screening for the compound comprising
(i) adding an active sulfhydryl compound to a first plurality of fungal or yeast cells which express zinc finger proteins,
(ii) subsequently adding the test compound,
(iii) incubating the fungal or yeast cells in the presence of the test compound and the active sulfhydryl compound, and
(iv) taking a first measurement of the zinc ejection resulted from the reaction of the compound and the zinc finger by a zinc florescence assay.

30. The pharmaceutical composition of claim 29, wherein the method further comprises
(v) adding a test compound to a second plurality of fungal or yeast cells which express zinc finger proteins,
(vi) incubating fungal or yeast cells with the test compound,
(vii) taking a second measurement of the zinc ejection resulted from the reaction of the compound and the zinc finger by a zinc florescence assay, and
(viii) comparing the first and the second measurements of the zinc ejection to designate the test compound as effective or non-effective for binding to or associating with fungal zinc finger motif,
wherein the active sulfhydryl compound is added at a higher concentration than is used for the test compound.

31. The pharmaceutical composition of claim 30, wherein the test compound is added at a concentration varying from about 0.01 nM to about 50 μM.

32. A method of treating or preventing a fungal or yeast infection comprising administering to a patient a pharmaceutical composition comprising an effective amount of a compound effective for binding to or associating with fungal zinc finger motif in a method for screening for the compound comprising
(i) adding an active sulfhydryl compound to a first plurality of fungal or yeast cells which express zinc finger proteins,
(ii) subsequently adding the test compound,
(iii) incubating the fungal or yeast cells in the presence of the sulfhydryl compound and the test compound,
(iv) taking a first measurement of the viability of the fungal or yeast cells,
(v) adding a test compound to a second plurality of fungal or yeast cells which express zinc finger proteins,
(vi) incubating fungal or yeast cells in the presence of the test compound,
(vii) taking a second measurement of the viability of the fungal or yeast cells, and
(viii) comparing the first and the second measurement of the viability of the fungal or yeast cells to designate the test compound as effective or non-effective for binding to or associating with fungal zinc finger motif,
wherein the active sulfhydryl compound is added at a higher concentration than is used for the test compound.

33. A method of treating or preventing a fungal or yeast infection comprising administering to a patient a pharmaceutical composition comprising an effective amount of a compound effective for binding to or associating with fungal zinc finger motif proteins in a method for screening for the compound comprising
(i) adding an active sulfhydryl compound to a first plurality of fungal or yeast cells which express zinc finger proteins,
(ii) subsequently adding the test compound,
(iii) incubating the fungal or yeast cells in the presence of the test compound and the active sulfhydryl compound,
(iv) taking a first measurement of the zinc ejection resulted from the reaction of the compound and the zinc finger by a zinc florescence assay,
(v) adding a test compound to a second plurality of fungal or yeast cells which express zinc finger proteins,
(vi) incubating fungal or yeast cells with the test compound, and
(vii) taking a second measurement of the zinc ejection resulted from the reaction of the compound and the zinc finger by a zinc florescence assay, and
(viii) comparing the first and the second measurements of the zinc ejection to designate the test compound as effective or non-effective for binding to or associating with fungal zinc finger motif,
wherein the active sulfhydryl compound is added at a higher concentration than is used for the test compound.
